(19) United States
(12) Patent Application Publication
(10) Pub. No.: US 2025/0241629 A1
(43) Pub. Date: Jul. 31, 2025

(54) TISSUE SAMPLING DEVICES AND HANDLES THEREOF, AND METHODS OF USING THE SAME

(71) Applicant: Bard Peripheral Vascular, Inc., Franklin Lakes, NJ (US)

(72) Inventors: Cristian Clavijo, Lehi, UT (US); Baiju Babu, Chandler, AZ (US)

(73) Assignee: Bard Peripheral Vascular, Inc., Franklin Lakes, NJ (US)

(21) Appl. No.: 18/848,387

(22) PCT Filed: Apr. 7, 2022
(86) PCT No.: PCT/US2022/023863

§ 371 Date: Sep. 18, 2024

(51) Int. Cl. A61B 10/02

(52) U.S. Cl. CPC A61B 10/0275; A61B 2010/0208

(57) ABSTRACT

Embodiments herein are directed to a tissue sampling device. The tissue sampling device includes a housing and a sub-frame positioned within the housing. A stylet hub is movably coupled to the sub-frame and has an outer surface and a resilient member extending from the outer surface. The resilient member is configured to transition between an unflexed state and a flexed state. The stylet hub is movable between a cocked position and an activated position along a central axis with respect to the sub-frame. A cannula hub is movably coupled to the sub-frame and movable between the cocked position the activated position the central axis with respect to the sub-frame. The resilient member is actuated between the flexed state and the unflexed state when the resilient member is in contact with a portion of the sub-frame as a function of a kinetic energy input exerted on the resilient member.

### TECHNICAL FIELD

The present specification generally relates to tissue sampling devices and, more specifically, to handheld automated tissue sampling devices.

### BACKGROUND

Some practitioners that perform core tissue sampling procedures prefer a self-contained handheld device over that of a large console system. A self-contained handheld biopsy device typically includes a stylet having a pointed distal tip and a side port proximal to the distal tip configured to receive tissue that will be severed to form a tissue sample. The shaft of the stylet may be in the form of a rod. A cutter cannula is positioned coaxial with and external to the stylet to sever the tissue received in the side port of the stylet.

One type of self-contained handheld biopsy device is fully disposable spring powered core needle biopsy devices. However, known spring powered core needle biopsy devices include many components to properly function. For example, known devices use silicone bumpers to keep the stylet hub away from a cannula hub during cocking, but they compress significantly during activation allowing the stylet hub to successfully disengage the cannula hub.

### SUMMARY

In one aspect, a handle is provided. The handle includes a housing, a sub-frame positioned within the housing, a stylet hub and a cannula hub. The stylet hub is moveably coupled to the sub-frame. The stylet hub has an outer surface and a resilient member extending from the outer surface. The resilient member configured to transition between an unflexed state and a flexed state. The stylet hub is movable between a cocked position and a released position along a central axis with respect to the sub-frame. The sub-frame having a portion configured to engage the resilient member when the stylet hub is in a predetermined position. The cannula hub is movably coupled to the sub-frame. The cannula hub being movable between a cocked position and a released position along the central axis with respect to the sub-frame. The resilient member of the stylet hub is configured to transition between the flexed state and the unflexed state dependent on a kinetic energy and a potential energy input exerted onto the resilient member when the resilient member is in contact with the portion of the sub-frame.

In another aspect, a tissue sampling device is provided. The tissue sampling device includes a handle a cannula, and a stylet. The handle includes a housing, a sub-frame, a cannula hub, and a stylet hub. The sub-frame is positioned within the housing. The stylet hub is movably coupled to the sub-frame. The stylet hub has an outer surface and a resilient member extending from the outer surface. The resilient member is configured to transition between an unflexed state and a flexed state. The stylet hub is movable between a cocked position and a released position along a central axis with respect to the sub-frame. The cannula hub is movably coupled to the sub-frame. The cannula hub is movable between a cocked position and a released position along the central axis with respect to the sub-frame. The sub-frame having a portion configured to engage the resilient member when the stylet hub is in a predetermined position. The resilient member of the stylet hub is configured to transition between the flexed state and the unflexed state dependent on a kinetic energy and a potential energy input exerted onto the resilient member when the resilient member is in contact with the portion of the sub-frame. The cannula has a first end and an opposite second end. A lumen extends between the first end and the second end. The second end is coupled to the cannula hub. The cannula extends along a cannula axis. The stylet is disposed within the lumen of the cannula having a tip portion and an opposite terminating end. The terminating end is coupled to the stylet hub. A tissue receiving recess positioned between the tip portion and the terminating end. In the released position, the stylet hub is actuated to move along the central axis and the stylet moves along the cannula axis, the cannula hub is then actuated to move along the central axis and the cannula moves along the cannula axis.

A method of using a tissue sampling device is provided. The method includes cocking a cannula hub and a stylet hub via a cocking slider such that the cannula hub and the stylet hub are held to store an energy, positioning a cutting edge of a cutting cannula at a target tissue gathering area, and actuating the stylet hub via the cocking slider or a trigger device to advance within a sub-frame of a handle of the tissue sampling device such that the stylet hub makes contact with a portion of the cannula hub to release and advance the cannula hub into a released position. The stylet hub includes a resilient member extending from an outer surface and transitions between an unflexed state and a flexed state dependent on a kinetic energy and a potential energy input exerted on the resilient member such that in the flexed state, the stylet hub is permitted to make contact with the portion of the cannula hub to release and advance the cannula hub into the released position, and in the unflexed state, the stylet hub is inhibited from advancing into a predetermined space such that clearance is provided to cock the cannula hub into a cocked position.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a perspective view of an example tissue cutting biopsy device, according to one or more embodiments shown and described herein;

FIG. 2A schematically depicts a side view of a prior art stylet and stylet hub, according to one or more embodiments shown and described herein;

FIG. 2B schematically depicts a front view of the prior art stylet and stylet hub of FIG. 2A, according to one or more embodiments shown and described herein;

FIG. 3 schematically depicts an isolated side view of a tissue cutting cannula and a tissue acquiring inner stylet of the example tissue cutting device of FIG. 1, according to one or more embodiments shown and described herein;

FIG. 4A schematically depicts an isolated cross sectional view of the example tissue cutting biopsy device of FIG. 1 taken from the line 4-4 with a cannula hub and a stylet hub in a cocked position, according to one or more embodiments shown and described herein;

FIG. 4B schematically depicts the isolated cross sectional view of the tissue cutting device of FIG. 4A with the cannula hub in the cocked position and the stylet hub in a released position according to one or more embodiments shown and described herein;

FIG. 4C schematically depicts the isolated cross sectional view of the example tissue cutting device of FIG. 4A with the cannula hub releasing from the cocked position and the stylet hub in the released position according to one or more embodiments shown and described herein;

FIG. 4D schematically depicts the isolated cross sectional view of the example tissue cutting device of FIG. 4A with the cannula hub and the stylet hub both in the released position according to one or more embodiments shown and described herein;

FIG. 5 schematically depicts an isolated perspective view of a first aspect stylet hub of the example tissue cutting device of FIG. 1, according to one or more embodiments shown and described herein;

FIG. 6 schematically depicts an isolated perspective view of a second aspect stylet hub of the example tissue cutting device of FIG. 1, according to one or more embodiments shown and described herein;

FIG. 7 schematically depicts an isolated perspective view of a third aspect stylet hub of the example tissue cutting device of FIG. 1, according to one or more embodiments shown and described herein;

FIG. 8 schematically depicts an isolated perspective view of a fourth aspect stylet hub of the example tissue cutting device of FIG. 1, according to one or more embodiments shown and described herein;

FIG. 9 schematically depicts an isolated cross sectional view of a resilient member of a stylet hub of the example tissue cutting device of FIG. 1 in an unflexed state, according to one or more embodiments shown and described herein;

FIG. 10 schematically depicts the isolated cross sectional view of the resilient member of the stylet hub of the example tissue cutting device of FIG. 9 in a flexed state, according to one or more embodiments shown and described herein;

FIG. 11 schematically depicts an illustrative graphical representation of an energy graph, according to one or more embodiments shown and described herein; and

FIG. 12 depicts a flow diagram of an illustrative method of gathering a tissue sample with the example tissue cutting device according to one or more embodiments shown and described herein.

### DETAILED DESCRIPTION

Embodiments described herein generally relate to a hand-held core needle biopsy device or tissue sampling device. The core needle biopsy device is used for the obtaining of a small sample from a soft tissue within a patient or subject. “Hand-held” means that the core needle biopsy device may be operated while being held in the hand (such as by one hand) of a user. The core needle biopsy device may include a concentric needle set which is inserted into the subject and activated for sample collection. During activation, a tissue sample is captured by the concentric needle set which may then be sent for pathology examination, for example. It is typical in a clinical procedure of this type to perform a plurality of sample acquisitions in the same subject to ensure good coverage of the region.

Tissue sampling occurs by first cocking the tissue sampling device by moving a stylet hub and a cannula hub into a respective cocked position and then activating the stylet hub to move into a released position, which in turn moves the cannula hub to move into a released position. The movement of the stylet hub into the released position and then the cannula hub into the released position results in a serial translational movement of the concentric needle set (e.g., a cannula and stylet). Both the stylet and the cannula are spring-loaded. A user activates the core needle biopsy device to move the stylet hub into the released position by pressing a fire button (or other user input device), which causes the stylet hub to accelerate the stylet forward into the target location of the tissue. The stylet tip geometry consists of an indentation or recess for tissue collection. Once the stylet is fully extended, the cannula hub is moved from the cocked position into the released position, which causes the cannula to accelerate forward. The cannula cuts the tissue which gets captured inside the stylet indentation.

The cocking and subsequent activation for sample acquisition is operated via an internal mechanism. For example, a resilient member may be positioned on a stylet hub and may move or transition between an unflexed state and a flexed state. During cannula cocking, the resilient member of the stylet hub is in an unflexed state to make contact with a portion of a sub-frame, which in turn maintains the stylet hub away from the cannula hub allowing the cannula hub to move into a cocked position. As such, the resilient member inhibits movement of the stylet hub into a predetermined space thereby providing a clearance space between the stylet hub and the cannula hub allowing the cannula hub to move from the released state into the cocked position.

During activation, the stylet hub accelerates towards the cannula hub via a coefficient of the spring, to make contact with the portion of the sub-frame causing the resilient member to move or transition from the unflexed state into the flexed state dependent on the kinetic and potential energies generated by the spring, such that the stylet hub advances to make contact with the cannula hub and a disengagement of the cannula hub occurs. Simultaneously, the stylet hub may drive the stylet beyond an end portion of the cannula into a tissue gathering position such that the tissue is captured inside the stylet indentation of the stylet. Further, the release of kinetic energy from the stylet hub moves the resilient member into the flexed state such that the stylet hub is able to make contact with the cannula hub and to advance the cannula to advance beyond the stylet indentation of the stylet into a sample acquisition position thereby cutting off any tissue not in the stylet indentation of the stylet.

As such, the embodiments described herein provide advantages over existing devices, such as the removal or need for additional components beyond the stylet hub itself to achieve successful cocking and sample acquisition. Reduction of additional components is advantageous because it lowers cost and improves manufacturability such as time to assemble the device, complexity of the device and design/quality related testing intricacy, and the like. Furthermore, embodiments of the resilient member may require overall less material in the stylet hub than existing solutions, which results in further manufacturing cost reductions. In addition, elimination of components removes potential points of failure for such devices.

Various embodiments of core needle biopsy devices and methods of use thereof are described in detail herein.

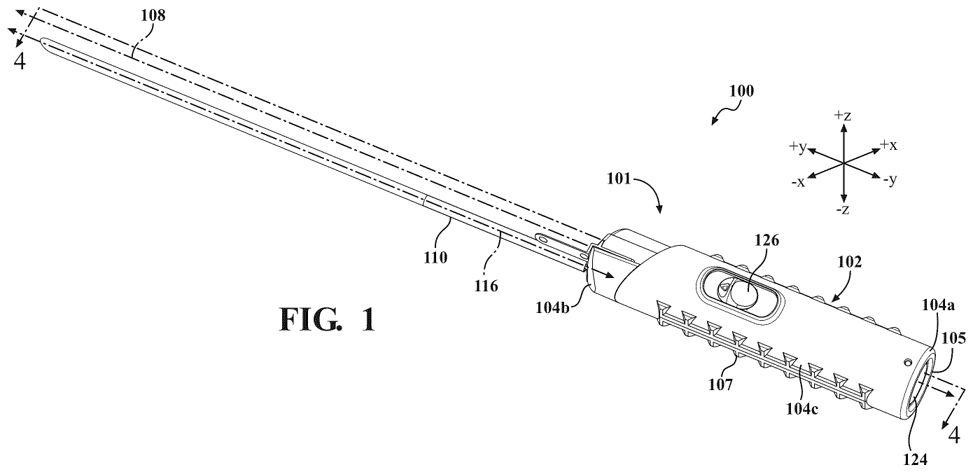

As used herein, the term “lateral direction” refers to the forward-rearward direction of the biopsy device (i.e., in the +/−Y-direction depicted in FIG. 1). The term “longitudinal direction” refers to the cross-biopsy device direction (i.e., in the +/−X-direction depicted in FIG. 1), and is transverse to the lateral direction. The term “vertical direction” or “up” or “above” refer to the upward-downward direction of the biopsy device (i.e., in the +/−Z-direction depicted in FIG. 1).

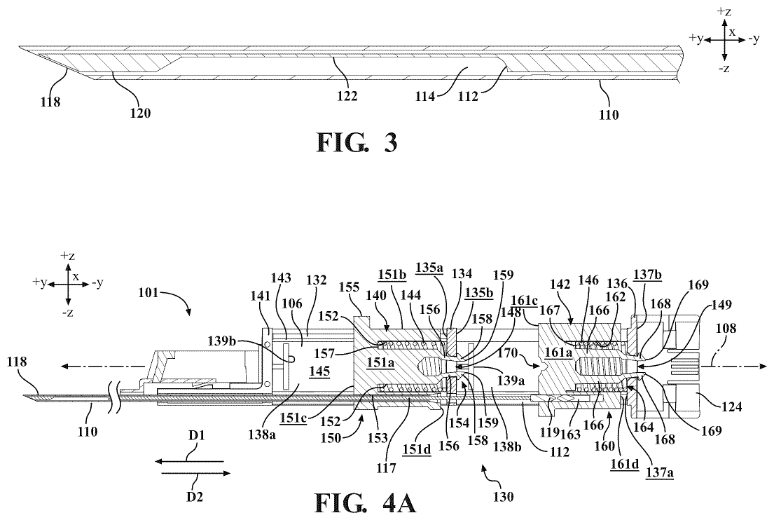

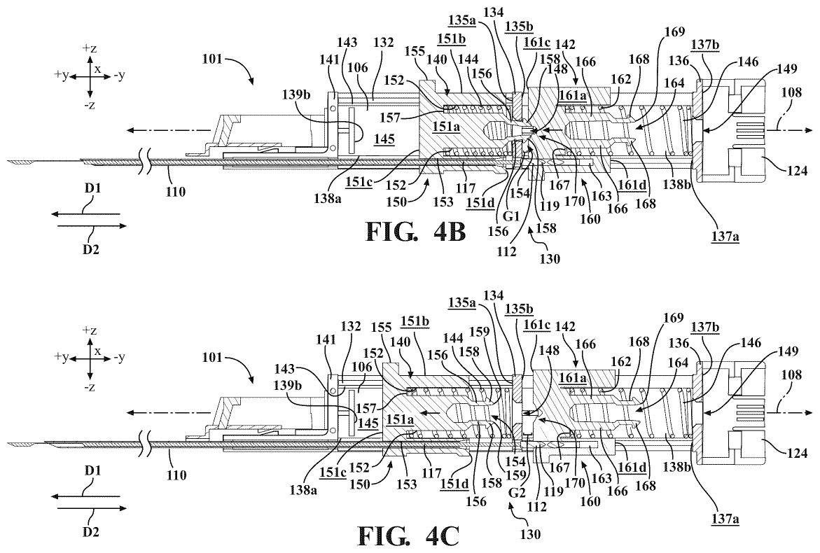

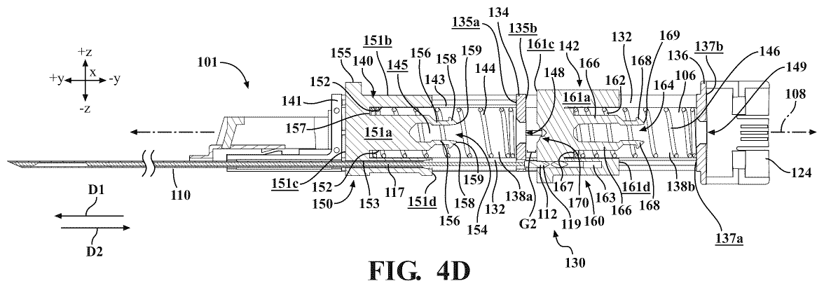

Referring now to the drawings, and more particularly, to FIGS. 1, 3, and 4A-4D, an example tissue sampling device 100 or core needle biopsy device is schematically depicted. The example tissue sampling device 100 includes a handle 101 having a housing 102 which may include a proximal end 104a, a distal end 104b, and a sidewall 104c. The sidewall 104c may define an interior chamber 106 having a central axis, depicted by the dashed arrow line 108. For ease of understanding the disclosure, FIG. 1 schematically depicts an exterior view of the example tissue sampling device 100, FIG. 3 schematically depicts an isolated side view of a tissue cutting cannula 110 and a tissue acquiring inner stylet 112, while FIGS. 4A-4D schematically depicts cross sectional views of the example tissue sampling device 100 in various operating conditions.

The housing 102 is depicted herein as a cylindrical shape. This is non-limiting and the housing 102 may take on any shape, such as a hexagonal, rectangular, octagonal, and/or the like. The proximal end 104a may include an annular wall that defines a circular opening 105. A plurality of raised ridges 107 may be formed on and extend along the housing 102. The plurality of raised ridges 107 may provide a texture for additional grip on the housing 102.

Extending or projecting distally from distal end 104b of the housing 102 is the tissue cutting cannula 110 and the tissue acquiring inner stylet 112 (illustrated in FIGS. 3 and 4A-4D). Referring to FIG. 3, the tissue cutting cannula 110 may be a hollow elongated member with a lumen 114 extending therethrough. The tissue cutting cannula 110 has a cutting edge 118 that is sharpened or beveled to slice through tissue. The tissue cutting cannula 110 may be formed of stainless steel, tungsten, or other suitable material for cutting through tissue.

The tissue acquiring inner stylet 112 may be slidably received within the lumen 114 of the tissue cutting cannula 110. That is, the tissue acquiring inner stylet 112 may be shaped and sized to dimensionally slide within the lumen 114 of the tissue cutting cannula 110. The tissue cutting cannula 110 and tissue acquiring inner stylet 112 may be coaxial with respect to an axis 116 depicted as a dashed arrow line in FIG. 1. Axis 116 may be parallel to, and/or may be radially offset from, the central axis 108.

The tissue acquiring inner stylet 112 may be an elongated member that may be configured with a tip 120 dimensionally shaped to penetrate skin. For example, the tip 120 may be sharpened or pointed. The tissue acquiring inner stylet 112 may further include a tissue receiving recess 122, or notch formed as a side recess proximal to the tip 120. The tissue acquiring inner stylet 112 may be formed of stainless steel, tungsten, a polymeric material or other suitable material.

Referring to FIG. 1, user operational features of the example tissue sampling device 100 that are accessible at the exterior of the housing 102 may include a trigger device 124 and a cocking slider 126. The trigger device 124 may be located at the proximal end 104a such as within the circular opening 105 of the housing 102, or any other location. The cocking slider 126 may be located along the sidewall 104c of the housing 102, such as on the distal top side of the housing 102, or any other location. The trigger device 124 and the cocking slider 126 may be operationally connected to one another. For example, the trigger device 124 and the cocking slider 126 may be operationally connected to one another via an elongated connector bar or other connecting device (not shown). It should be appreciated that there may be a plurality of triggers (e.g., the cocking slider 126 may act as a trigger and the trigger device 124) at various locations, each of the plurality triggers assist to accommodate various user's preference. Some users, such as radiologists and surgeons, may prefer a trigger on the top of the device, such as cocking slider 126, while others, such as urologist may prefer a trigger on the rear of the device, such as the trigger device 124.

It should be appreciated that the example tissue sampling device 100 is movable between a cocked position and a released position, as discussed in greater detail herein. The cocked position may be where components of the example tissue sampling device 100 are in a position to store an energy and the released position may be where the stored energy is used such that components of the example tissue sampling device 100 gather tissue samples, as discussed in greater detail herein.

For example, the tissue acquiring inner stylet 112 is movable between a retracted position, where the tip 120 of the tissue acquiring inner stylet 112 may be positioned within the lumen 114 such that the cutting edge 118 of the tissue cutting cannula 110 extends distally to the tissue receiving recess 122, as best illustrated in FIG. 4A, and a tissue gathering position, where the tissue receiving recess 122 of the tissue acquiring inner stylet 112 may be extended distally beyond the cutting edge 118 along the axis 116, as best illustrated in FIG. 4B. The tissue cutting cannula 110 is movable between a retracted position where the tip 120 of the tissue acquiring inner stylet 112 may be covered by the cutting edge 118 and the lumen 114, as best illustrated in FIG. 4A, and a sample acquisition position where the tip 120 and the tissue receiving recess 122 of the tissue acquiring inner stylet 112 may be moved to extend beyond the cutting edge 118, as best illustrated in FIGS. 4B to 4C.

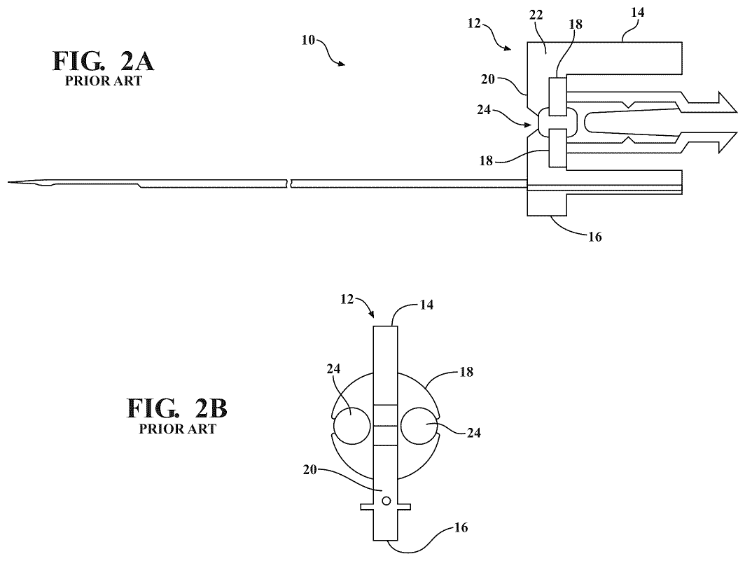

Now referring to FIGS. 2A-2B, an isolated side view of a stylet 10 and a stylet hub 12 of a prior art biopsy instrument is schematically depicted. As depicted, the stylet hub 12 of the prior art biopsy instrument includes upper and lower trailing arms 14, 16. A disk-shaped member 18 is mounted to a rear edge 20 of an upright 22, between the upper and lower trailing arms 14, 16. A pair of bumpers 24 are mounted one on either side of the disk-shaped member 18. As illustrated, in this device, the bumpers 24 are dumbbell-shaped, with enlarged head portions connected by a reduced neck portion. Slots are formed in the disk-shaped member 18 of the stylet hub 12 dimensioned to receive the reduced neck portion of the bumpers 24. In this conventional device, the bumpers 24 are made of silicone having a hardness of 65-75 on the Shore A scale. As such, the bumpers 24 are subjected to wear over time which creates issues for functionality, requires the additional components to retain bumpers 24 during compression and expansion, and the bumpers 24 and additional components to retain the bumpers 24 are subject to failure.

In the released position, the stylet hub 12 of the conventional device has been driven forward, the bumpers 24 deform upon contact, such that the stylet hub 12 continues to advance, which creates a contact with a cannula hub (not shown). In turn, the contact of the stylet hub 12 may collapse locking tangs and disengaging flanges of the cannula hub such that the cannula hub is now free to be driven forward by a spring (not shown). The bumpers 24 then exert a rearward force against the stylet hub 12 as they return to their normal state, displacing the stylet hub 12 slightly rearward such that the upright 22 of the stylet hub 12 is spaced sufficiently from the cannula hub. As such, the disk-shaped member 18 and each of the bumpers 24 are required to provide the clearance necessary to position the cannula hub into the cocked position and to disengage the cannula hub when in the cocked position. As such, the bumpers 24 are subjected to wear over time which creates issues for functionality, requires the additional components to retain bumpers 24 during compression and expansion, and the bumpers 24 and additional components to retain the bumpers 24 are subject to failure.

Now referring to FIGS. 4A-4D and 9-10, the example tissue sampling device 100 may further include a sub-frame 130 positioned in the interior chamber 106 of the housing 102. As discussed in greater detail herein, the sub-frame 130 may be used to guide a cannula hub 140 and a stylet hub 142 between a cocked position and a released position. Additionally, the sub-frame 130 engages with a resilient member 180 of the stylet hub 142 such that the resilient member 180 transitions between a flexed state and an unflexed state, as discussed in greater detail herein.

The sub-frame 130 may be formed as into halves, such as a first sub-frame portion 132 and an opposite second sub-frame portion (not shown). Each sub-frame portion may be tubular, or half-moon shaped, so that together, they form a cylindrical shape. As used herein, the terms “first” and “second” are used merely as relative terms for ease of explaining the present disclosure. As such, only the first sub-frame portion 132 will be explained in detail herein as the first sub-frame portion 132 and the opposite second sub-frame portion are mirror images of one another and are received within the housing 102 of the handle 101.

The first sub-frame portion 132 may have a wall 143 that is semi-tubular. The wall 143 may have a surface 145 that is arcuate and that defines a distal interior chamber 138a between a separator wall 134 and a distal end wall 141, and a proximal interior chamber 138b defined between the separator wall 134 and a proximal end wall 136. As such, the distal interior chamber 138a and the proximal interior chamber 138b may be divided by the separator wall 134. The separator wall 134 may include a forward surface 135a and an opposite rear surface 135b and include a bore 148 extending through the forward surface 135a and the rear surface 135b. In some embodiments, the bore 148 may be positioned to be aligned with the central axis 108. That is, the bore 148 may be coaxially aligned along the central axis 108. It should be appreciated that this is a non-limiting example, and the bore 148 may be offset from the central axis 108.

The proximal end wall 136 may include an inner surface 137a and an opposite outer surface 137b. Further, the proximal end wall 136 may include a bore 149 extending through the inner surface 137a and the outer surface 137b. In some embodiments, the bore 149 may be positioned to be aligned with the central axis 108. That is, the bore 149 may be coaxially aligned along the central axis 108. It should be appreciated that this is a non-limiting example, and the bore 149 may be offset from the central axis 108. Further, in some embodiments, the bore 148 and the bore 149 may be coaxially aligned with respect to one another, whether aligned with the central axis 108 or not. In other embodiments, the bore 148 and the bore 149 may be offset from one another.

Still referring to FIGS. 4A-4D and 9-10, the proximal interior chamber 138b may include a proximal protrusion 139a extending from the surface 145 of the wall 143 into the proximal interior chamber 138b between the separator wall 134 and the proximal end wall 136. The distal interior chamber 138a may include a distal protrusion 139b extending from the surface 145 of the wall 143 into the distal interior chamber 138a between the separator wall 134 and the distal end wall 141. As discussed in greater detail herein, the proximal protrusion 139a make contact or engage with the resilient member 180 of the stylet hub 142 such that the resilient member 180 transitions between the flexed state and the unflexed state based on the amount of force applied to the resilient member 180. As such, the positioning of the proximal protrusion 139a may impact or change the positioning of the stylet hub 142 and the resilient member 180 making contact with the proximal protrusion 139a.

The cannula hub 140 may be fixedly coupled to a proximal end 117 of the tissue cutting cannula 110. The cannula hub 140 may be positioned within the distal interior chamber 138a and movable between the separator wall 134 and the distal end wall 141, as discussed in greater detail herein. The cannula hub 140 may further include a cannula hub body 150. The cannula hub body 150 may define an outer surface 151a, an opposite inner surface 151b to define a thickness, an upper terminating surface 151c, and an opposite lower terminating surface 151d. A post 155 may extend outwards from the thickness from by the outer surface 151a and the opposite inner surface 151b. The post 155 may be captured and coupled to the cocking slider 126 of the housing 102 (FIG. 1) to move the cannula hub 140 from the actuated state into the cocked position, as discussed in greater detail herein.

At least one inner channel 152 may be formed within the cannula hub body 150 and extend from the lower terminating surface 151d towards the upper terminating surface 151c without extending through the upper terminating surface 151c. As such, in some embodiments, the cross-section of the cannula hub body 150 may generally be U-shaped. In other embodiments, the cross-section of the cannula hub body 150 may be another shape, such as, for example, T-shaped, M-shaped, V-shaped, or the like, without departing from the scope of the present disclosure.

The at least one inner channel 152 may include an end wall 157 positioned near or adjacent to the upper terminating surface 151c. The at least one inner channel 152 may be sized and dimensionally shaped to receive portions of the cannula spring 144 such that, as the cannula spring 144 extends and compresses, as discussed in greater detail herein, the cannula hub 140 moves within the distal interior chamber 138a.

The cannula hub body 150 may further include a pair of locking members 154 that are formed proximal to the cannula hub body 150 and from the end wall 157. The pair of locking members 154 may extend from the end wall 157 towards and beyond the lower terminating surface 151d. Each one of the pair of locking members 154 may be formed as elongated cantilevered arms 156. Each of the elongated cantilevered arms 156 may include respective outwardly facing latching portions 158 at a distal end. Each of the outwardly facing latching portions 158 may be hooks that include outer ramped surfaces 159 that converge in distal direction D2 (e.g., the outer ramped surfaces 159 taper in the distal direction D2). In the cocked position, the outwardly facing latching portions 158 are received and engaged with the bore 148 of the separator wall 134 to couple the cannula hub 140 to the separator wall 134, as best illustrated in FIGS. 4A-4B, and as discussed in greater detail herein.

Still referring to FIGS. 4A-4D and 9-10, in some embodiments, the cannula hub body 150 may be formed (e.g., molded or otherwise manufactured) from acetal containing compounds, nylon, polyetheretherketone, polybutylene terephthalate, and/or the like. Further, in some embodiments, the cannula hub body 150 may be formed via injection molding principles. In other embodiments, the cannula hub body 150 may be formed via additive manufacturing processes.

In some embodiments, the upper terminating surface 151c may be a planar surface. In other embodiments, the upper terminating surface 151c may be angled, arcuate, curvilinear, and/or the like. The upper terminating surface 151c may include a cutting cannula receiving aperture 153 that receives the proximal end of the tissue cutting cannula 110. The cutting cannula receiving aperture 153 may be a pass through opening that extends and passes through the cannula hub body 150 between the upper terminating surface 151c and the lower terminating surface 151d.

The wall 143 of the distal interior chamber 138a receives and supports the cannula hub 140 with a cannula spring 144 interposed between the cannula hub 140 and the separator wall 134. The cannula spring 144 may be formed of stainless steel, a polymeric material, or other suitable material. Further, the spring rating of the cannula spring 144 may be equal or vary based on the application.

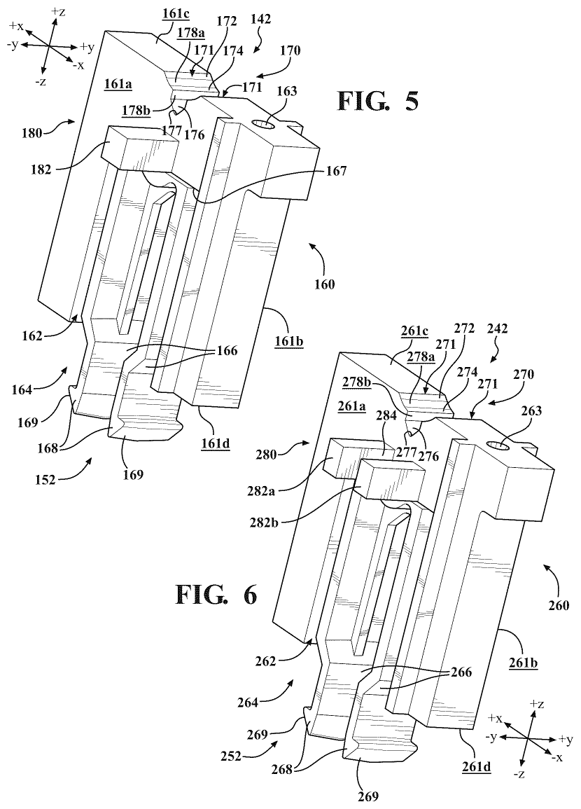

Still referring to FIGS. 4A-4D and 9-10 and also now referring to FIGS. 5-8, the stylet hub 142 may be fixedly coupled to a proximal end 117 of tissue acquiring inner stylet 112. The stylet hub 142 may be positioned within the proximal interior chamber 138b and movable between the separator wall 134 and the proximal end wall 136, as discussed in greater detail herein.

The stylet hub 142 may further include a stylet hub body 160. The stylet hub body 160 may define an outer surface 161a, an opposite inner surface 161b to define a thickness, an upper terminating surface 161c, and a lower terminating surface 161d. A post 165 may extend outwards from the thickness from by the outer surface 161a and the opposite inner surface 161b. The post 165 may be captured and coupled to the cocking slider 126 of the housing 102 (FIG. 1) to move the stylet hub 142 from the actuated state into the cocked position, as discussed in greater detail herein. In some embodiments, there may be more than one cocking slider 126 of the housing 102 (FIG. 1), one to move the cannula hub 140 from the actuated state into the cocked position and one to move the stylet hub 142 from the actuated state into the cocked position. In other embodiments, both the cannula hub 140 and the stylet hub 142 are coupled to the cocking slider 126 and the user may selectively move the cannula hub 140 from the actuated state into the cocked position and the stylet hub 142 from the actuated state into the cocked position.

At least one inner channel 162 may be formed within the stylet hub body 160 and extend from the lower terminating surface 161d towards the upper terminating surface 161c without extending into the upper terminating surface 161c. As such, in some embodiments, the stylet hub body 160 may generally be U-shaped. In other embodiments, the stylet hub body 160 may be another shape, such as, for example, T-shaped, M-shaped, V-shaped, or the like, without departing from the scope of the present disclosure.

The at least one inner channel 162 may include an end wall 167 positioned near or adjacent to the upper terminating surface 161c. The at least one inner channel 162 may receive portions of the stylet spring 146 such that, as the stylet spring 146 extends and compresses, as discussed in greater detail herein, the stylet hub 142 moves within the proximal interior chamber 138b.

The stylet hub body 160 may further include a pair of locking members 164 that are formed proximal to the stylet hub body 160 and from the end wall 167. The pair of locking members 164 may extend from the end wall 167 towards and beyond the lower terminating surface 161d. Each one of the pair of locking members 164 may be formed as elongated cantilevered arms 166. Each of the elongated cantilevered arms 166 may include respective outwardly facing latching portions 168. Each of the outwardly facing latching portions 168 may be hooks that include outer ramped surfaces 169 that converge in distal direction D2 (e.g., the outer ramped surfaces 169 taper in the distal direction D2). In the cocked position, the outwardly facing latching portions 168 are received and engaged with the bore 149 of the proximal end wall 136, as best illustrated in FIG. 4A, and as discussed in greater detail herein.

In some embodiments, the stylet hub body 160 may be formed (e.g., molded or otherwise manufactured) from acetal containing compounds, nylon, polyetheretherketone, polybutylene terephthalate, and/or the like. Further, in some embodiments, the stylet hub body 160 may be formed via injection molding principles. In other embodiments, the stylet hub body 160 may be formed via additive manufacturing processes.

Still referring to FIGS. 4A-4D and 5-10, in some embodiments, the upper terminating surface 161c may be a planar surface that includes a cannula hub actuation feature 170. In other embodiments, the upper terminating surface 161c may be angled, arcuate, curvilinear, and/or the like. The upper terminating surface 161c may include a tissue acquiring inner stylet receiving aperture 163 that receives the distal end 119 of the tissue acquiring inner stylet 112. The tissue acquiring inner stylet receiving aperture 163 may extend through the stylet hub body 160 with the tissue acquiring inner stylet receiving aperture 163 positioned at the upper terminating surface 151c and extending towards the lower terminating surface 161d. In some embodiments, the tissue acquiring inner stylet receiving aperture 163 may pass through the lower terminating surface 161d.

The cannula hub actuation feature 170 may generally extend from the upper terminating surface 161c towards the lower terminating surface 161d and interacts with the cannula hub 140 to release the cannula hub 140 from the cocked position to released position. For example, the cannula hub actuation feature 170 may include a stepped shape.

The cannula hub actuation feature 170 may include pair of sidewalls 171 that each include a first or leading step portion 172, a second or following step portion 174 and a trench 176 having an apex 177. The following step portion 174 may extend deeper into the stylet hub body 160 compared to the leading step portion 172. The following step portion 174 may terminate at the trench 176. The following step portion 174 may include a step surface 178a and a side step surface 178b such that the following step portion 174 may extend deeper into the stylet hub body 160 in a direction away from the upper terminating surface 161c and towards the lower terminating surface 161d. The side step surface 178b may be perpendicular to the step surface 178a. The leading step portion 172 may be angled with respect to the step surface 178a of the following step portion 174. Further, the apex 177 pf the trench 176 may be angled as an inverse apex positioned away from the leading step portion 172 and the following step portion 174. As such, the trench 176 may generally be a V-shape. This is non-limiting and the trench 176 may be a U-shape, or any other shape capable of performing the functionality as described in greater detail herein.

The cannula hub actuation feature 170 may act as a guide to move the outwardly facing latching portions 158 and the outer ramped surfaces 159 of the pair of locking members 154 of the cannula hub 140 from engagement with the bore 148 in the cocked position, as discussed in greater detail herein.

The wall 143 of the proximal interior chamber 138b receives and support the stylet hub 142 with a stylet spring 146 interposed between the stylet hub 142 and the proximal end wall 136. The stylet spring 146 may be formed of stainless steel, a polymeric material, or other suitable material. Further, the spring rating of the stylet spring 146 may be equal or vary based on the application.

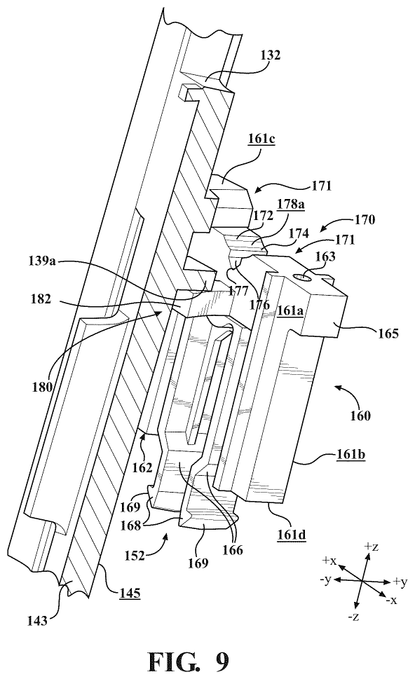

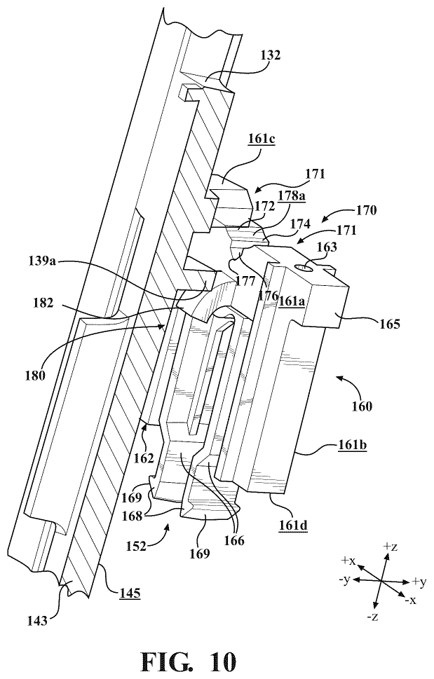

Now referring to FIGS. 5 and 10-11, the resilient member 180 may extend from the outer surface 161a of the stylet hub 142 in the system lateral direction (i.e., in the +/−Y direction). The resilient member 180 may be a tab member that acts as a transient hinge such that the tab member may be movable between the unflexed state, as best illustrated in FIG. 9, and a flexed state, as best illustrated in FIG. 10, and as discussed in greater detail herein. In the unflexed state, the resilient member 180 prevents a movement of the stylet hub 142 into a predetermined space between the separator wall 134 and the upper terminating surface 161c of the stylet hub 142. Additionally, in the unflexed state, contact between the proximal protrusion 139a and the resilient member 180 may move the stylet hub 142 out of the predetermined space between the separator wall 134 and the upper terminating surface 161c of the stylet hub 142.

In the flexed state, the resilient member 180 may transition as the transient hinge when contact with the proximal protrusion 139a is made, permitting the stylet hub 142 to move into the predetermined space. That is, in the unflexed state, the resilient member 180 is in contact with the proximal protrusion 139a of the sub-frame 130 to prevent the stylet hub 142 from advancing in the direction D1 beyond a predetermined position with respect to the separator wall 134. This allows for the necessary clearance to permit the cannula hub 140 to be moved into the cocked position, as best illustrated in FIG. 4B, such that the respective outwardly facing latching portions 158 of the cannula hub 140 may engage with the rear surface 135b of the separator wall 134.

In the aspects described with respect to FIG. 5, the resilient member 180 may be a shaped tab member 182, such as a cuboid, cylinder, cone, prism, and/or the like. It should be understood that the present disclosure is not limited to uniformly shapes and the tab member may be non-uniformly shaped. The shaped tab member 182 extends outwardly from the outer surface 161a in the lateral direction (i.e., in the +/±Y-direction). In some embodiments, the shaped tab member 182 may be positioned between the upper terminating surface 161c and the at least one inner channel 162. In embodiments, the shaped tab member 182 may be positioned between the cannula hub actuation feature 170 and the end wall 167 of the at least one inner channel 162. Further, in this aspect, the shaped tab member 182 may be aligned with the apex 177 of the trench 176 of the cannula hub actuation feature 170. As such, in these embodiments, the shaped tab member 182 may be positioned to be centered between the pair of locking members 164 and extend in a direction perpendicular to the direction that the pair of locking members 164 extend in the at least one inner channel 162.

In other embodiments, the shaped tab member 182 is not limited to its positioning and may be positioned to extend from the outer surface 161a or the inner surface 161b of the stylet hub 142 and may be positioned anywhere between the upper terminating surface 161c and the at least one inner channel 162. Further, it should be appreciated and understood that the resilient member 180 is not limited to the shapes of the shaped tab member 182 depicted, and may be any shape capable of moving between the unflexed state and the flexed state and capable of preventing an advancement of the stylet hub 142 when in the unflexed state.

The resilient member 180 may be formed with a predetermined force threshold to transition the resilient member 180 between the flexed state and the unflexed state when contact is made with the proximal protrusion 139a of the sub-frame 130. That is, the amount of energy exerted onto the resilient member 180 at the moment of contact with the proximal protrusion 139a of the sub-frame 130 transitions the resilient member 180 between the flexed state, as best illustrated in FIG. 10, and the unflexed state, as best illustrated in FIG. 9. For example, when the resilient member 180 is exposed to generally low energy inputs (e.g., kinetic energies less than 0.05 Joules), the resilient member 180 may maintain the unflexed state. For kinetic energies higher than 0.05 Joules, the resilient member 180 may transition to the flexed state. It should be appreciated that these are merely examples and the resilient member 180 may change the threshold force values through the geometric shape, size, materials, and the like. As such, in the released position, the resilient member 180 may experience more than the predetermined higher energy input from the energy stored within the stylet spring 146 pushing the stylet hub 142 through the distal interior chamber 138a such that the resilient member 180 engages or makes contact with the proximal protrusion 139a of the sub-frame 130 with kinetic and potential energies greater than the amount required to transition the resilient member 180 into the flexed state. When the energy from the stylet spring 146 is exhausted, the kinetic energy and potential energy applied to the resilient member 180 is less than the amount required to transition the resilient member 180 into the flexed state such that the resilient member 180 transitions into or remains in the unflexed state.

Still referring to FIGS. 5 and 9-10, in the flexed state, the stylet hub 142 may be able to move freely within the proximal interior chamber 138b until the upper terminating surface makes contact with the rear surface 135b of the separator wall 134. In the unflexed state, the resilient member 180 may hold or maintain a gap between the upper terminating surface 161c and the rear surface 135b of the separator wall 134, as best illustrated in FIGS. 4C-4D, when the resilient member 180 is positioned against the proximal protrusion 139a. That is, in the unflexed state, the resilient member 180 makes contact with the proximal protrusion 139a of the sub-frame 130 to move or prevent movement of the stylet hub 142 to provide the gap between the upper terminating surface 161c and the rear surface 135b of the separator wall 134, as best illustrated in FIGS. 4C-4D. As such, the gap between the rear surface 135b of the separator wall 134 and the upper terminating surface 161c of the stylet hub 142 is larger when the resilient member 180 is in the unflexed state, illustrated by the bracket G2 in FIGS. 4C-4D that the gap between the rear surface 135b of the separator wall 134 and the upper terminating surface 161c of the stylet hub 142 when the resilient member 180 is in the flexed state, illustrated by the bracket G1 in FIG. 4B. It should be understood that this clearance is necessary for the cannula hub 140 to be moved from the released position into the cocked position, as discussed in greater detail herein. As such, it should be appreciated that the resilient member 180 is transitioned from the unflexed state to the flexed state as a function of the energy input (e.g., the kinetic energy and the potential energy) placed on the resilient member 180 from the driving force of the stylet spring 146 and the contact between the resilient member 180 and the proximal protrusion 139a.

It should also be appreciated that in the flexed state, the resilient member 180 allows for the stylet hub 142 to advance forward within the proximal interior chamber 138b in the direction D1 such that the cannula hub actuation feature 170 of the stylet hub 142 receives the outwardly facing latching portions 158 of the cannula hub 140.

In some embodiments, the resilient member 180 may be formed or molded with the stylet hub body 160. As such, the resilient member 180 may be a monolithic structure with the stylet hub body 160. In other embodiments, the resilient member 180 is separate from the body and coupled to the stylet hub body 160. In these embodiments, the resilient member 180 is coupled to the body using known techniques such as adhesive, welding, epoxy, fasteners, and/or the like.

Now referring to FIG. 6, another embodiment of a stylet hub 242 is schematically depicted. It is understood that the stylet hub 242 is similar to the stylet hub 142 with the exceptions of the features described herein. As such, like features will use the same reference numerals with a prefix “2” for the reference numbers. As such, for brevity reasons, these features will not be described again.

In the present embodiment, a resilient member 280 extends from the outer surface 261a of the stylet hub 242 in the lateral direction (i.e., in the +/−Y-direction). The resilient member 280 may be a pair of spaced apart shaped tab members 282a, 282b extending outwardly from the outer surface 261a. In some embodiments, the pair of spaced apart shaped tab members 282a, 282b may be similarly shaped. In other embodiments, the pair of spaced apart shaped tab members 282a, 282b may be dissimilarly dimensioned. It should be understood that the present disclosure is not limited to uniformly shaped tab members and each or both of the tab members may be non-uniformly shaped. In the depicted embodiment, each of the pair of spaced apart shaped tab members 282a, 282b may have a width, and/or a thickness less than the resilient member 180 (FIG. 5). Each of the pair of spaced apart shaped tab members 282a, 282b provide a transient hinge that are each movable between the unflexed state and the flexed state.

In the embodiment described in FIG. 6, a space 284 between the pair of spaced apart shaped tab members 282a, 282b may align with the inverse apex 277 of the trench 276 of the stepped feature portion 270. As such, the space 284 between the pair of spaced apart shaped tab members 282a, 282b may be positioned to be centered between the pair of locking members 264 and extend in a direction perpendicular to the direction that the pair of locking members 264 extend in the at least one inner channel 262. In some embodiments, the pair of spaced apart shaped tab members 282a, 282b may transition between the flexed state and the unflexed state simultaneously. In other embodiments, the pair of spaced apart shaped tab members 282a, 282b may independently transition between the flexed state and the unflexed state.

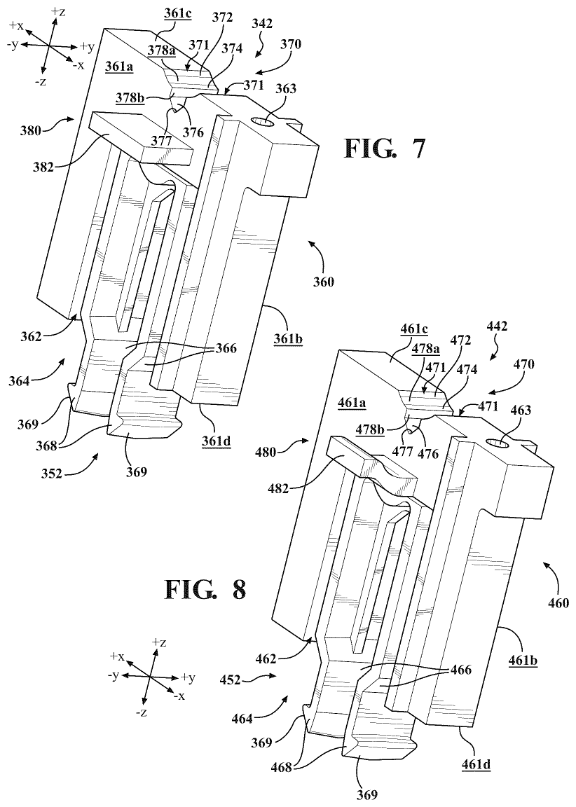

Now referring to FIG. 7, another embodiment of a stylet hub 342 is schematically depicted. It is understood that the stylet hub 342 is similar to the stylet hub 142 with the exceptions of the features described herein. As such, like features will use the same reference numerals with a prefix “3” for the reference numbers. As such, for brevity reasons, these features will not be described again.

A resilient member 380 extends from the outer surface 361a of the stylet hub 342 in the lateral direction (i.e., in the +/−Y-direction). The resilient member 380 may be a shaped tab member 382 having less of a thickness in the longitudinal direction (i.e., in the +/−X-direction) than the distance the resilient member extends from the outer surface 361a in the lateral direction (i.e., in the +/−Y-direction). As such, in a non-limiting example, the shaped tab member 382 may be a flat cuboid shape. In some embodiments, the shaped tab member 382 may be positioned between the upper terminating surface 361c and the at least one inner channel 362. In embodiments, the shaped tab member 382 may be positioned between the cannula hub actuation feature 370 and the end wall 367 of the at least one inner channel 362. Further, in this aspect, the shaped tab member 382 may be aligned with the inverse apex of the trench 376 of the cannula hub actuation feature 370. As such, in these embodiments, the shaped tab member 382 may be positioned to be centered between the pair of locking members 364 and extend in a direction perpendicular to the direction that the pair of locking members 364 extend in the at least one inner channel 362.

Now referring to FIG. 8, yet another embodiment of a stylet hub 442 is schematically depicted. It is understood that the stylet hub 442 is similar to the stylet hub 142 with the exceptions of the features described herein. As such, like features will use the same reference numerals with a prefix “4” for the reference numbers. As such, for brevity reasons, these features will not be described again.

A resilient member 480 extends from the outer surface 361a of the stylet hub 442. The resilient member 480 may include a shaped tab member 482 that is a shaped tab member 482 extending outwardly from the outer surface 461a in the lateral direction (i.e., in the +/−Y-direction). For example, the shaped tab member 482 may be substantially s-shaped in the +/−X/Z plane of the depicted coordinate axes. In some embodiments, the shaped tab member 482 may be positioned between the upper terminating surface 461c and the at least one inner channel 462. In embodiments, the shaped tab member 482 may be positioned between the cannula hub actuation feature 470 and the end wall 467 of the at least one inner channel 462. Further, in this aspect, the shaped tab member 482 may be aligned with the inverse apex of the trench 476 of the cannula hub actuation feature 470. As such, in these embodiments, the shaped tab member 482 may be positioned to be centered between the pair of locking members 464 and extend in a direction perpendicular to the direction that the pair of locking members 464 extend in the at least one inner channel 462.

It should be appreciated that, in some embodiments, the various resilient members 180, 280, 380, 480 of FIGS. 5-8 are differently shaped to create different or varying force profiles to transition between the flexed state and the unflexed state. As such, the resilient member 480 of FIG. 8 may use less potential and kinetic energy generated by the stylet spring 146 to transition between the flexed state and the unflexed state than the resilient member 180 of FIG. 5. This is a non-limiting example as are the shapes of the various resilient members 180, 280, 380, 480 illustrated in FIGS. 5-8.

Now referring back to FIGS. 1 and 4A-4D, to operate the core needle biopsy device 100, a user may cock the example tissue sampling device 100. As used herein, “cocking” of core needle biopsy device/tissue sampling device is the process by which each of the cannula spring 144 and the stylet spring 146, respectively and independently, are compressed (e.g., either simultaneously or individually) and held in the compressed state (cocked position) to store energy (e.g., kinetic energy and potential energy) that will be released during a firing operation. In the compressed state, the outwardly facing latching portions 158 of the cannula hub 140 are received and engaged with the bore 148 of the separator wall 134 to couple the cannula hub 140 to the separator wall 134, as best illustrated in FIGS. 4A-4B. Further, in the compressed state, the outwardly facing latching portions 168 of the stylet hub 142 are received and engaged with the bore 149 of the proximal end wall 136 to couple the stylet hub 142 to the proximal end wall 136, as best illustrated in FIG. 4A.

As noted above, the tissue cutting cannula 110 and the tissue acquiring inner stylet 112 may be cocked simultaneously or, the tissue cutting cannula 110 and the tissue acquiring inner stylet 112 may be cocked sequentially. With the example tissue sampling device 100 cocked, the tissue cutting cannula 110 may be introduced into the subject (e.g., such as a soft tissue region of the subject) and advanced until the tissue acquiring inner stylet 112 lies adjacent to the target tissue to be sampled.

In the cocked position, with respect to FIGS. 1 and 4A, the outwardly facing latching portions 168 of the elongated cantilevered arms 166 of the stylet hub 142 extend through the bore 149 in the proximal end wall 136 engaging with the outer surface 137b of the proximal end wall 136 to retain the stylet hub 142 in the cocked position. With the stylet hub 142 in the cocked position, the stylet spring 146 is compressed between the end wall 167 of the at least one inner channel 162 of the stylet hub 142 and the inner surface 137a of the proximal end wall 136. As such, the outwardly facing latching portions 168 of the elongated cantilevered arms 166 of the stylet hub 142 may act as a first latch for releasably holding the stylet spring 146 in an energized mode (e.g., with a build-up of kinetic and potential energies).

Similarly, in the cocked position, the outwardly facing latching portions 158 and the outer ramped surfaces 159 of the elongated cantilevered arms 156 of the cannula hub 140 extend through the bore 148 in the separator wall 134 such that the outwardly facing latching portions 158 and a back surface of the outer ramped surfaces 159 engage with a rear surface 135b of the separator wall 134. With the cannula hub 140 in the cocked position, the cannula spring 144 is compressed between the end wall 157 of the at least one inner channel 152 of the cannula hub 140 and the forward surface 135a of the separator wall 134. As such, the outwardly facing latching portions 158 of the elongated cantilevered arms 156 of the cannula hub 140 may act as a second latch for releasably holding the cannula spring 144 in an energized mode.

With the stylet hub 142 and cannula hub 140 both in the cocked position, both the tissue acquiring inner stylet 112 and the tissue cutting cannula 110 are in the retracted position. As such, in some embodiments, the tissue receiving recess 122 of the tissue acquiring inner stylet 112 may be positioned within the lumen 114 and may be covered by the cutting edge 118 of the tissue cutting cannula 110. In some embodiments, the tip 120 of the tissue acquiring inner stylet 112 may protrude slightly from the cutting edge 118 of the tissue cutting cannula 110, with the tissue receiving recess 122 of the tissue acquiring inner stylet 112 positioned within the lumen 114 and proximate to the cutting edge 118 of the tissue cutting cannula 110.

Referring now to FIGS. 1 and 4B, once the cutting edge 118 of tissue cutting cannula 110 has been properly positioned at the target tissue gathering site of the subject, the user actuates the example tissue sampling device 100 by actuating (such as by depressing) either the trigger device 124 or the cocking slider 126. Actuation of the trigger device 124 or cocking slider 126 may cause the trigger device 124 to exert a forward force in the direction D1 to release the outwardly facing latching portions 168 of the elongated cantilevered arms 166 of the stylet hub 142 from the bore 149 of the proximal end wall 136. Alternatively, advancing the cocking slider 126 may exert a forward force in the direction D1 against the elongated connector bar (not shown) to release the outwardly facing latching portions 168 of the elongated cantilevered arms 166 of the stylet hub 142 from the bore 149 of the proximal end wall 136.

Upon release of the stylet hub 142, the stylet hub 142 may be driven in the direction D1 toward the separator wall 134 under force provided by the stylet spring 146 such that the stylet spring exerts a kinetic energy and potential energy onto the stylet hub 142 and components thereof (e.g., the resilient member 180). The outwardly facing latching portions 158 of the elongated cantilevered arms 156 of the cannula hub 140 may be received in the cannula hub actuation feature 170 of the stylet hub 142. In response to the stylet hub 142 driven in the direction D1 toward the separator wall 134 under force provided by the stylet spring 146, the tissue acquiring inner stylet 112 may move in direction D1 along axis 116 from the retracted position to the tissue gathering position where the tissue receiving recess 122 of the tissue acquiring inner stylet 112 is extended beyond the cutting edge 118.

Now referring to FIGS. 4C and 10, when the resilient member 180 of the stylet hub 142 makes contact with the proximal protrusion 139a, at or near the separator wall 134, the resilient member 180 transitions from the unflexed state to the flexed state such that the stylet hub 142 may advance and the cannula hub actuation feature 170 of the stylet hub 142 may make contact and collapse the outwardly facing latching portions 158 of the cannula hub 140. The contact and collapse of the outwardly facing latching portions 158 may be in a direction towards one another such that the outwardly facing latching portions 158, when in or near the trench 176, disengage with separator wall 134. Further, in this position, a diameter of the outwardly facing latching portions 158 may be smaller than the bore 148 of the separator wall 134, thereby releasing the cannula hub 140 from the separator wall 134 and permitting the outwardly facing latching portions 158 to pass through the bore 148. The cannula hub 140 then advances forward in the direction D1 driven by the cannula spring 144, as best illustrated in FIGS. 4C-4D.

By advancing the cannula hub 140, the cutting edge 118 and the lumen 114 of the tissue cutting cannula 110 is advanced to slide over the tissue receiving recess 122 and may cut or remove any excess tissue such that tissue is permitted to prolapse into the tissue receiving recess 122 of the tissue acquiring inner stylet 112. That is, the cannula hub 140 advances in the direction D1 until the upper terminating surface 151c of the cannula hub 140 makes contact with the distal end wall 141. In this position, the tissue cutting cannula 110 has again advanced over the tissue acquiring inner stylet 112 and sheared off the tissue, which has prolapsed into the tissue receiving recess 122 of the tissue acquiring inner stylet 112, capturing it within the lumen 114 of the tissue cutting cannula 110.

As such, now the tissue acquiring inner stylet 112 is in the tissue gathering position and the tissue cutting cannula 110 is in the sample acquisition position, where the cutting edge 118 is moved to extend beyond the tissue gathering position of the tissue acquiring inner stylet 112 such that the tissue receiving recess 122 are covered by the cutting edge 118 and the lumen 114. As such, in this position, the relationship between the tissue cutting cannula 110 and the tissue acquiring inner stylet 112 is similar to that when the tissue cutting cannula 110 and the tissue acquiring inner stylet 112 are in the retracted position when the cannula hub 140 and the stylet hub 142 are in the energized state.

Upon completion of sample retrieval, the user may then withdraw the tissue cutting cannula 110 and the tissue acquiring inner stylet 112 from the subject with the tissue sample positioned within the tissue receiving recess 122.

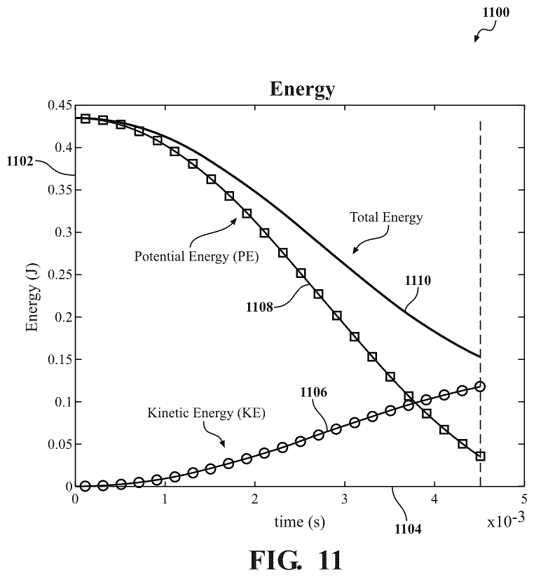

Now referring to FIG. 11, an illustrative graphical representation of an energy graph 1100 is schematically depicted. The energy graph 1100 is an energy (Joules) versus time (seconds) graph with respect to the resilient member 180 (FIG. 5). The energy is plotted on an ordinate 1102 and the time is plotted on an abscissa 1104. As shown, a kinetic energy 1106, depicted as a line having circles, increases with time while a potential energy 1108, depicted as a line having diamonds, lowers over time. As such, a total energy 1110, depicted by a solid line, varies as a function of both the potential and kinetic energies 1106, 1108. As depicted, the kinetic energy and the potential energy intersect at 0.1 Joules and time almost at 4 seconds×10⁻³. As discussed above, an arbitrary threshold for the resilient member 180 (FIG. 5) is set to one-half of that intersection, or 0.05 Joules of required kinetic energy 1106 to move the resilient member from the unflexed state to the flexed state, as discussed herein. It should be appreciated that the threshold may be varied and that this is a non-limiting example.

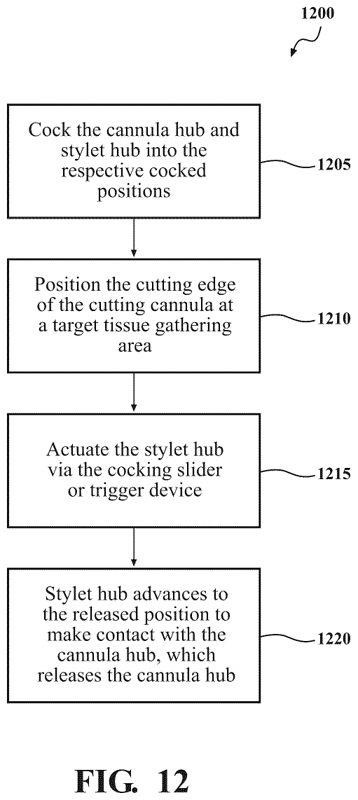

Referring now to FIGS. 1, 3, 4A-4D and 12, a flow diagram that graphically depicts an illustrative method 1200 for gathering a tissue sample using the tissue sampling device 100 is provided. Although the steps associated with the blocks of FIG. 12 will be described as being separate tasks, in other embodiments, the blocks may be combined or omitted. Further, while the steps associated with the blocks of FIG. 12 will described as being performed in a particular order, in other embodiments, the steps may be performed in a different order.

At block 1205, the cannula hub 140 is cocked into the cocked position and the stylet hub 142 is cocked into the cocked position via a cocking slider 126. In the cocked positions, both the cannula hub 140 and the stylet hub 142 are held to store an energy such as the energy (e.g., kinetic energy and potential energy) of the cannula spring 144 and the stylet spring 146, respectively. At block 1210, the cutting edge 118 of the cutting cannula 110 is positioned at a target tissue gathering area. At block 1215, the stylet hub 142 is actuated via the cocking slider 126 or trigger device 124 to advance to the released position. The actuating of the stylet hub 142 advances the stylet hub 142 within a sub-frame 130 of the handle 101 of the tissue sampling device 100 such that the stylet hub 142 makes contact with a portion of the cannula hub 140 to release and advance the cannula hub into the released position, at block 1210.

To make contact with the portion of the cannula hub 140, the resilient member 180 extending from the outer surface 161a of the stylet hub body 160 transitions from the unflexed state into the flexed state as a function of a kinetic energy input exerted on the resilient member 180 from the driving force of the stylet spring 146 and the contact between the resilient member 180 and the proximal protrusion 139a.

As such, in the flexed state, the stylet hub 142 is permitted to make contact with the portion of the cannula hub 140 to release and advance the cannula hub 140 into the released position. In the unflexed state, the stylet hub 142 is inhibited from advancing into the predetermined space such that clearance is provided to move the cannula hub 140 into the cocked position.

It should be appreciated that the example core needle biopsy device 100 produce desirable results over existing solutions, such as the removal of any additional components beyond the stylet hub itself to achieve successful cocking and sample acquisition. A reduction of additional components is advantageous because it lowers cost of goods, time to assemble the device, complexity of the device and design/quality related testing intricacies, and the like.

Embodiments of the present disclosure may be further described with respect to the following numbered clauses:
1. A handle comprising: a housing; a sub-frame positioned within the housing; a stylet hub movably coupled to the sub-frame, the stylet hub having an outer surface and a resilient member extending from the outer surface, the resilient member configured to transition between an unflexed state and a flexed state, the stylet hub being movable between a cocked position and a released position along a central axis with respect to the sub-frame; the sub-frame comprising a portion configured to engage the resilient member when the stylet hub is in a predetermined position; and a cannula hub being movably coupled to the sub-frame, the cannula hub being movable between a cocked position and a released position along the central axis with respect to the sub-frame; wherein the resilient member of the stylet hub is configured to transition between the flexed state and the unflexed state dependent on a kinetic energy and a potential energy input exerted onto the resilient member when the resilient member is in contact with the portion of the sub-frame.
2. The handle of any preceding clause, wherein when the kinetic energy and the potential energy input exerted onto the resilient member is less than a predetermined amount of energy when the resilient member is in contact with the portion of the sub-frame, the resilient member remains in the unflexed state.
3. The handle of any preceding clause, wherein when the resilient member is in the unflexed state and in contact with the portion of the sub-frame, the resilient member provides a space between the stylet hub and the cannula hub allowing the cannula hub to move into and remain in the cocked position.
4. The handle of any preceding clause, wherein when the kinetic energy and the potential energy input exerted onto the resilient member is more than a predetermined amount of energy when the resilient member is in contact with the portion of the sub-frame, the resilient member transitions into the flexed state
5. The handle of any preceding clause, wherein: the stylet hub comprises a cannula hub actuation feature for releasing the cannula hub from the cocked position, and when the resilient member is in the flexed state and in contact with the portion of the sub-frame, the cannula hub actuation feature contacts with the cannula hub thereby actuating the cannula hub into the released position.
6. The handle of any preceding clause, wherein the stylet hub further comprises: an inner surface positioned opposite of the outer surface to define a thickness of a body, an upper terminating surface and an opposite lower terminating surface; at least one inner channel extending from a lower terminating surface in a direction towards the upper terminating surface and terminating before the upper terminating surface; and a cannula hub actuation feature positioned along and extending into the upper terminating surface.
7. The handle of any preceding clause, wherein the cannula hub actuation feature further comprises: a pair of spaced apart sidewalls, each one of the pair of spaced apart sidewalls comprising: a leading step portion; a following step portion; and a trench, wherein the following step portion is angled to the leading step portion and extends deeper into the body of the stylet hub than the leading step portion in a vertical direction in a direction away from the upper terminating surface and towards the lower terminating surface.
8. The handle of any preceding clause, wherein the resilient member comprises a cuboid tab member.
9. The handle of any preceding clause, wherein the resilient member comprises a pair of spaced apart cuboid tab members.
10. The handle of any preceding clause, wherein the resilient member comprises a flat cuboid tab member.
11. The handle of any preceding clause, wherein the resilient member comprises a curvilinear flat cuboid tab member.
12. A tissue sampling device, comprising: a handle comprising: a housing; a sub-frame positioned within the housing; a stylet hub movably coupled to the sub-frame, the stylet hub having an outer surface and a resilient member extending from the outer surface, the resilient member configured to transition between a unflexed state and a flexed state, the stylet hub movable between a cocked position and a released position along a central axis with respect to the sub-frame; the sub-frame comprising a portion configured to engage the resilient member when the stylet hub is in a predetermined position; and a cannula hub movably coupled to the sub-frame, the cannula hub movable between the cocked position and the released position along the central axis with respect to the sub-frame; wherein the resilient member of the stylet hub is configured to transition between the flexed state and the unflexed state dependent on a kinetic energy and a potential energy input exerted onto the resilient member when the resilient member is in contact with the portion of the sub-frame, a cannula having a first end and an opposite second end, a lumen extending between the first end and the second end, the second end coupled to the cannula hub, the cannula extending along a cannula axis; and a stylet disposed within the lumen of the cannula having a tip portion and an opposite terminating end, the terminating end is coupled to the stylet hub, a tissue receiving recess positioned between the tip portion and the terminating end, wherein, when moving from the cocked position to the released position, the stylet hub moves along the central axis and the stylet moves along the cannula axis and the cannula hub is then actuated to move along the central axis and the cannula moves along the cannula axis.
13. The tissue sampling device of any preceding clause, wherein when the kinetic energy and the potential energy input exerted onto the resilient member is less than a predetermined amount of energy when the resilient member is in contact with the portion of the sub-frame, the resilient member remains in the unflexed state.
14. The tissue sampling device of any preceding clause, wherein when the kinetic energy and the potential energy input exerted onto the resilient member from the stylet is less than the predetermined amount of energy when the resilient member is in contact with the portion of the sub-frame, the resilient member transitions or remains in the unflexed state.
15. The tissue sampling device of any preceding clause, wherein when the resilient member is in the unflexed state and in contact with the portion of the sub-frame, the resilient member provides a space between the stylet hub and the cannula hub thereby allowing the cannula hub to move into the cocked position.
16. The tissue sampling device of any preceding clause, wherein when the kinetic energy and the potential energy input exerted onto the resilient member from the stylet is more than a predetermined amount of energy when the resilient member is in contact with the portion of the sub-frame, the resilient member transitions into the flexed state.
17. The tissue sampling device of any preceding clause, wherein: the stylet hub comprises a cannula hub actuation feature for releasing the cannula hub from the cocked position, and when the resilient member is in the flexed state and in contact with the portion of the sub-frame, the cannula hub actuation feature contacts with the cannula hub thereby actuating the cannula hub into the released position.
18. The tissue sampling device of any preceding clause, wherein the stylet hub further comprises: an inner surface positioned opposite of the outer surface to define a thickness of a body; an upper terminating surface and an opposite lower terminating surface; and a cannula hub actuation feature positioned along and extending into the upper terminating surface, the cannula hub actuation feature comprising: a pair of spaced apart sidewalls, each one of the pair of spaced apart sidewalls comprising: a leading step portion; a following step portion; and a trench, wherein the following step portion is angled to the leading step portion and extends deeper into the body of the stylet hub than the leading step portion in a vertical direction in a direction away from the upper terminating surface and towards the lower terminating surface and wherein the following step portion is angled to the leading step portion.
19. The tissue sampling device of any preceding clause, wherein the resilient member is a cuboid tab member.
20. The tissue sampling device of any preceding clause, wherein the resilient member is a pair of spaced apart cuboid tab members.
21. The tissue sampling device of any preceding clause, wherein the resilient member is a flat cuboid tab member.
22. The tissue sampling device of any preceding clause, wherein the resilient member is a curvilinear flat cuboid tab member.
23. A method of gathering a tissue sample using a tissue sampling device, the method comprising: cocking a cannula hub and a stylet hub via a cocking slider such that the cannula hub and the stylet hub are held to store an energy; positioning a cutting edge of a cutting cannula at a target tissue gathering area; and actuating the stylet hub via the cocking slider or a trigger device to advance within a sub-frame of a handle of the tissue sampling device such that the stylet hub makes contact with a portion of the cannula hub to release and advance the cannula hub into a released position, wherein the stylet hub comprises a resilient member extending from an outer surface and transitions between an unflexed state and a flexed state dependent on a kinetic energy and a potential energy input exerted on the resilient member such that in the flexed state, the stylet hub is permitted to make contact with the portion of the cannula hub to release and advance the cannula hub into the released position and, in the unflexed state, the stylet hub is inhibited from advancing into a predetermined space such that clearance is provided to cock the cannula hub into a cocked position.
24. The method of any preceding clause, wherein the kinetic energy and the potential energy input exerted on the resilient member is when the resilient member is in contact with a portion of the sub-frame.
25. The method of any preceding clause, wherein when the kinetic energy and the potential energy input exerted onto the resilient member is less than a predetermined amount of energy when the resilient member is in contact with the portion of the sub-frame, the resilient member remains in the unflexed state.
26. The method of any preceding clause, wherein when the kinetic energy and the potential energy input exerted onto the resilient member is more than a predetermined amount of energy when the resilient member is in contact with the portion of the sub-frame, the resilient member transitions into the flexed state.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A handle comprising:
a housing;
a sub-frame positioned within the housing;
a stylet hub movably coupled to the sub-frame, the stylet hub having an outer surface and a resilient member extending from the outer surface, the resilient member configured to transition between an unflexed state and a flexed state, the stylet hub being movable between a cocked position and a released position along a central axis with respect to the sub-frame;
the sub-frame comprising a portion configured to engage the resilient member when the stylet hub is in a predetermined position; and
a cannula hub being movably coupled to the sub-frame, the cannula hub being movable between a cocked position and a released position along the central axis with respect to the sub-frame;
wherein the resilient member of the stylet hub is configured to transition between the flexed state and the unflexed state dependent on a kinetic energy and a potential energy exerted onto the resilient member when the resilient member is in contact with the portion of the sub-frame.

2. The handle of claim 1, wherein when the kinetic energy and the potential energy input exerted onto the resilient member is less than a predetermined amount of energy when the resilient member is in contact with the portion of the sub-frame, the resilient member remains in the unflexed state.

3. The handle of claim 2, wherein when the resilient member is in the unflexed state and in contact with the portion of the sub-frame, the resilient member provides a space between the stylet hub and the cannula hub allowing the cannula hub to move into and remain in the cocked position.

4. The handle of claim 1, wherein when the kinetic energy and the potential energy input exerted onto the resilient member is more than a predetermined amount of energy when the resilient member is in contact with the portion of the sub-frame, the resilient member transitions into the flexed state.

5. The handle of claim 1, wherein:
the stylet hub comprises a cannula hub actuation feature for releasing the cannula hub from the cocked position, and
when the resilient member is in the flexed state and in contact with the portion of the sub-frame, the cannula hub actuation feature contacts with the cannula hub thereby actuating the cannula hub into the released position.

6. The handle of claim 1, wherein the stylet hub further comprises:
an inner surface positioned opposite of the outer surface to define a thickness of a body,
an upper terminating surface and an opposite lower terminating surface;
at least one inner channel extending from a lower terminating surface in a direction towards the upper terminating surface and terminating before the upper terminating surface; and
a cannula hub actuation feature positioned along and extending into the upper terminating surface.

7. The handle of claim 6, wherein the cannula hub actuation feature further comprises:
a pair of spaced apart sidewalls, each one of the pair of spaced apart sidewalls comprising:
a leading step portion;
a following step portion; and
a trench,
wherein the following step portion is angled to the leading step portion and extends deeper into the body of the stylet hub than the leading step portion in a vertical direction in a direction away from the upper terminating surface and towards the lower terminating surface.

8. The handle of claim 7, wherein the resilient member comprises at least one of a cuboid tab member, a pair of spaced apart cuboid tab members, a flat cuboid tab member or a curvilinear flat cuboid tab member.

9.-11. (canceled)

12. A tissue sampling device, comprising:
a handle comprising:
a housing;
a sub-frame positioned within the housing;
a stylet hub movably coupled to the sub-frame, the stylet hub having an outer surface and a resilient member extending from the outer surface, the resilient member configured to transition between a unflexed state and a flexed state, the stylet hub movable between a cocked position and a released position along a central axis with respect to the sub-frame;
the sub-frame comprising a portion configured to engage the resilient member when the stylet hub is in a predetermined position; and
a cannula hub movably coupled to the sub-frame, the cannula hub movable between the cocked position and the released position along the central axis with respect to the sub-frame;
wherein the resilient member of the stylet hub is configured to transition between the flexed state and the unflexed state dependent on a kinetic energy and a potential energy input exerted onto the resilient member when the resilient member is in contact with the portion of the sub-frame,
a cannula having a first end and an opposite second end, a lumen extending between the first end and the second end, the second end coupled to the cannula hub, the cannula extending along a cannula axis; and
a stylet disposed within the lumen of the cannula having a tip portion and an opposite terminating end, the terminating end is coupled to the stylet hub, a tissue receiving recess positioned between the tip portion and the terminating end,
wherein, when moving from the cocked position to the released position, the stylet hub moves along the central axis and the stylet moves along the cannula axis and the cannula hub is then actuated to move along the central axis and the cannula moves along the cannula axis.

13. The tissue sampling device of claim 12, wherein when the kinetic energy and the potential energy input exerted onto the resilient member is less than a predetermined amount of energy when the resilient member is in contact with the portion of the sub-frame, the resilient member remains in the unflexed state.

14. The tissue sampling device of claim 13, wherein when the kinetic energy and the potential energy input exerted onto the resilient member from the stylet is less than the predetermined amount of energy when the resilient member is in contact with the portion of the sub-frame, the resilient member transitions or remains in the unflexed state.

15. The tissue sampling device of claim 14, wherein when the resilient member is in the unflexed state and in contact with the portion of the sub-frame, the resilient member provides a space between the stylet hub and the cannula hub thereby allowing the cannula hub to move into the cocked position.

16. The tissue sampling device of claim 12, wherein when the kinetic energy and the potential energy input exerted onto the resilient member from the stylet is more than a predetermined amount of energy when the resilient member is in contact with the portion of the sub-frame, the resilient member transitions into the flexed state.

17. The tissue sampling device of claim 16, wherein:
the stylet hub comprises a cannula hub actuation feature for releasing the cannula hub from the cocked position, and
when the resilient member is in the flexed state and in contact with the portion of the sub-frame, the cannula hub actuation feature contacts with the cannula hub thereby actuating the cannula hub into the released position.

18. The tissue sampling device of claim 12, wherein the stylet hub further comprises:
an inner surface positioned opposite of the outer surface to define a thickness of a body;
an upper terminating surface and an opposite lower terminating surface; and
a cannula hub actuation feature positioned along and extending into the upper terminating surface, the cannula hub actuation feature comprising:
a pair of spaced apart sidewalls, each one of the pair of spaced apart sidewalls comprising:
a leading step portion;
a following step portion; and
a trench,
wherein the following step portion is angled to the leading step portion and extends deeper into the body of the stylet hub than the leading step portion in a vertical direction in a direction away from the upper terminating surface and towards the lower terminating surface and wherein the following step portion is angled to the leading step portion.

19. The tissue sampling device of claim 12, wherein the resilient member is at least one of a cuboid tab member, a pair of spaced apart cuboid tab members, a flat cuboid tab member, or a curvilinear flat cuboid tab member.

20.-22. (canceled)

23. A method of gathering a tissue sample using a tissue sampling device, the method comprising:
cocking a cannula hub and a stylet hub via a cocking slider such that the cannula hub and the stylet hub are held to store an energy;
positioning a cutting edge of a cutting cannula at a target tissue gathering area; and
actuating the stylet hub via the cocking slider or a trigger device to advance within a sub-frame of a handle of the tissue sampling device such that the stylet hub makes contact with a portion of the cannula hub to release and advance the cannula hub into a released position,
wherein the stylet hub comprises a resilient member extending from an outer surface and transitions between an unflexed state and a flexed state dependent on a kinetic energy and a potential energy input exerted on the resilient member such that in the flexed state, the stylet hub is permitted to make contact with the portion of the cannula hub to release and advance the cannula hub into the released position and, in the unflexed state, the stylet hub is inhibited from advancing into a predetermined space such that clearance is provided to cock the cannula hub into a cocked position.

24. The method of claim 23, wherein the kinetic energy and the potential energy input exerted on the resilient member is when the resilient member is in contact with a portion of the sub-frame.

25. The method of claim 24, wherein when the kinetic energy and the potential energy input exerted onto the resilient member is less than a predetermined amount of energy when the resilient member is in contact with the portion of the sub-frame, the resilient member remains in the unflexed state.

26. The method of claim 24, wherein when the kinetic energy and the potential energy input exerted onto the resilient member is more than a predetermined amount of energy when the resilient member is in contact with the portion of the sub-frame, the resilient member transitions into the flexed state.
